(19) United States
(12) Patent Application Publication
(10) Pub. No.: US 2020/0107754 A1
(43) Pub. Date: Apr. 9, 2020

(54) CYCLING-POSTURE ANALYZING SYSTEM AND METHOD

(71) Applicant: Institute For Information Industry, Taipei (TW)

(72) Inventors: Tse-Yu Lin, New Taipei City (TW); Yin-Yu Chou, Taitung County (TW); Shih-Yao Wei, Taipei City (TW)

(21) Appl. No.: 16/211,206

(22) Filed: Dec. 5, 2018

(30) Foreign Application Priority Data
Oct. 8, 2018 (TW) 107135396

(51) Int. Cl. A61B 5/11; A63B 69/16; A63B 71/06; A61B 5/103; A63B 24/00; A61B 5/00

(52) U.S. Cl. CPC A61B 5/1116; A61B 2560/0475; A63B 71/0622; A61B 5/1036; A63B 24/0062; A61B 5/1121; A61B 5/6823; A61B 5/6828; A61B 5/6829; A61B 5/4519; A61B 5/486; A63B 2071/063; A63B 2071/0658; A63B 2220/836; A63B 2220/56; A63B 2220/803; A63B 2225/50; A63B 2230/62; A61B 2503/10; A63B 2225/52; A63B 2024/0068; A61B 2562/0219; A63B 69/16

(57) ABSTRACT

A cycling-posture analyzing system and method are provided. The cycling-posture analyzing system includes a plurality of motion sensors, a pressure sensor and an electronic device. The plurality of motion sensors are disposed on a human body and are configured to detect a plurality of pieces of motion information. The pressure sensor is disposed at a plantar aspect of the human body and is configured to detect pressure information. The electronic device is configured to receive the plurality of pieces of motion information from the plurality of motion sensors, receive the pressure information from the pressure sensor, and use the plurality of pieces of motion information and the pressure information to determine cycling-posture type information according to a cycling-posture identification model.

### PRIORITY

This application claims priority to Taiwan Patent Application No. 107135396 filed on Oct. 8, 2018, which is hereby incorporated by reference in its entirety.

### FIELD

The present invention relates to a cycling-posture analyzing system and a cycling-posture analyzing method; and more particularly, the present invention uses a cycling-posture identification model to analyze data detected by motion sensors and a pressure sensor so as to determine cycling-posture type information of a bicycle.

### BACKGROUND

With the popularity of leisure activities, more and more people have joined the bicycle sport. People are turning their attention to know whether their cycling-posture is correct or not, so as to expect for achieving better efficiency and performance on bicycling or prevent the sports injuries caused by incorrect posture.

Currently, the analysis for the cycling-posture of bicyclists is often introduced by indoor image capturing, and movements of various parts of a human body are captured by a camera to analyze whether the cycling-posture is correct. However, due to the limitation of the image-capturing equipment, such kind of measuring manner cannot be promoted to measure the actual cycling state outdoors. Moreover, another method for analysis that is often used is measuring muscle states by sensors, and thereby obtain a measured surface electromyography (sEMG). However, human body could sweat copiously during the cycling, and such influences the adhesion ability and the detection precision of electrodes of the sensors, so it is hard to obtain precise analysis.

Accordingly, an urgent need exists in the art to provide a measuring method adapted for use in outdoor cycling to analyze the cycling-posture so as to provide riders with relevant suggestions, thereby improving the cycling performance of the riders.

### SUMMARY

To solve the aforesaid problem, provided are a cycling-posture analyzing system and a cycling-posture analyzing method.

The cycling-posture analyzing system may comprise a plurality of motion sensors, a pressure sensor and an electronic device. The plurality of motion sensors are disposed on a human body and configured to detect a plurality of pieces of motion information. The pressure sensor is disposed at a plantar aspect of the human body and configured to detect pressure information. The electronic device further comprises a transceiver and a processor, and the processor is electrically connected to the transceiver. The transceiver is configured to receive the plurality of pieces of motion information from the plurality of motion sensors and the pressure information from the pressure sensor. The processor is configured to determine cycling-posture type information according to the plurality of pieces of motion information and the pressure information and on the basis of a cycling-posture identification model.

The cycling-posture analyzing system may comprise a plurality of motion sensors, a pressure sensor and an electronic device. The plurality of motion sensors are disposed on a human body and configured to detect a plurality of pieces of motion information. The pressure sensor is disposed at a plantar aspect of the human body and configured to detect pressure information. The electronic device further comprises a transceiver. The transceiver is configured to: receive the plurality of pieces of motion information from the plurality of motion sensors and receive the pressure information from the pressure sensor; transmit the plurality of pieces of motion information and the pressure information to a cloud computing system so that the cloud computing system determines cycling-posture type information, muscle-group usage information and feedback information according to the plurality of pieces of motion information and the pressure information; and receive the cycling-posture type information, the muscle-group usage information and the feedback information from the cloud computing system.

A cycling-posture analyzing method for a cycling-posture analyzing system is also provided. The cycling-posture analyzing system comprises a plurality of motion sensors, a pressure sensor and an electronic device. The cycling-posture analyzing method comprises: detecting, by the plurality of motion sensors, a plurality of pieces of motion information, the plurality of motion sensors being disposed on a human body; detecting, by the pressure sensor, pressure information, the pressure sensor being disposed at a plantar aspect of the human body; receiving, by the electronic device, the plurality of pieces of motion information from the plurality of motion sensors and the pressure information from the pressure sensor; and determining, by the electronic device, cycling-posture type information according to the plurality of pieces of motion information and the pressure information on the basis of a cycling-posture identification model.

Further provided is a cycling-posture analyzing method for a cycling-posture analyzing system. The cycling-posture analyzing system comprises a plurality of motion sensors, a pressure sensor and an electronic device. The cycling-posture analyzing method comprises: detecting, by the plurality of motion sensors, a plurality of pieces of motion information, the plurality of motion sensors being disposed on a human body; detecting, by the pressure sensor, pressure information, the pressure sensor being disposed at a plantar aspect of the human body; receiving, by the electronic device, the plurality of pieces of motion information from the plurality of motion sensors and the pressure information from the pressure sensor; transmitting, by the electronic device, the plurality of pieces of motion information and the pressure information to a cloud computing system so that the cloud computing system determines cycling-posture type information, muscle-group usage information and feedback information according to the plurality of pieces of motion information and the pressure information; and receiving, by the electronic device, the cycling-posture type information, the muscle-group usage information and the feedback information from the cloud computing system.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a schematic view depicting a cycling-posture analyzing system according to a first embodiment;

FIG. 2 is a schematic view depicting angular information and pressure information in a cycling-posture analyzing system according to a second embodiment;

FIG. 3A is a schematic view depicting an electronic device according to a fourth embodiment;

FIG. 3B is a schematic view depicting an identification model according to the fourth embodiment;

FIG. 4A is a schematic view depicting an electronic device according to a fifth embodiment;

FIG. 4B is a schematic view depicting a feedback model according to the fifth embodiment;

FIG. 5 is a schematic view depicting an electronic device and a cloud computing system according to a sixth embodiment;

FIG. 6 is a schematic view depicting a cycling-posture analyzing method according to a seventh embodiment;

FIG. 7 is a schematic view depicting a cycling-posture analyzing method according to an eighth embodiment;

FIG. 8 is a schematic view depicting a cycling-posture analyzing method according to a ninth embodiment;

FIG. 9 is a schematic view depicting a cycling-posture analyzing method according to a tenth embodiment;

FIG. 10 is a schematic view depicting a cycling-posture analyzing method according to an eleventh embodiment; and

FIG. 11 is a schematic view depicting a cycling-posture analyzing method according to a twelfth embodiment.

### DETAILED DESCRIPTION

In the following description, the present invention will be explained with reference to certain example embodiments thereof. It shall be appreciated that these example embodiments are not intended to limit the present invention to any specific example, embodiment, environment, applications or particular implementations described in these example embodiments. Therefore, description of these example embodiments is only for purpose of illustration rather than to limit the present invention.

In the following embodiments and the attached drawings, elements unrelated to the present invention are omitted from depiction; and dimensional relationships among individual elements in the attached drawings are provided only for illustration, but not to limit the actual scale.

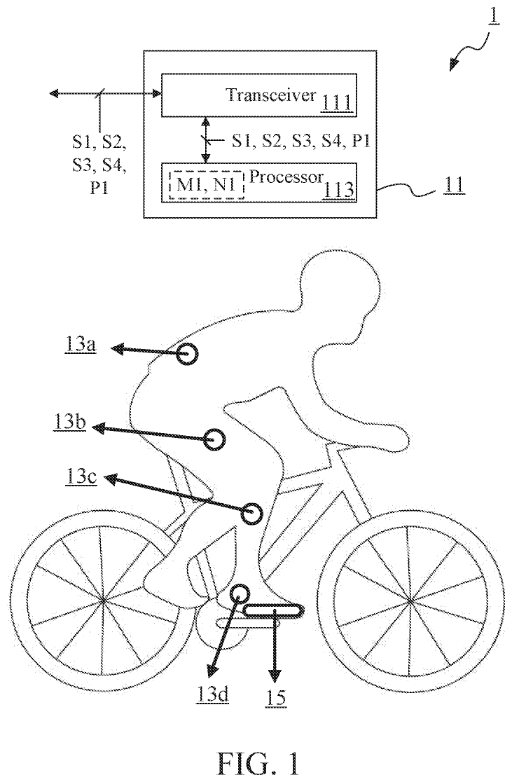

Please refer to FIG. 1 for a first embodiment of the present invention. FIG. 1 is a schematic view depicting a cycling-posture analyzing system 1. Herein, the cycling-posture analyzing system 1 comprises an electronic device 11, a plurality of motion sensors 13a to 13d and a pressure sensor 15, and the configuration thereof is as shown in FIG. 1. The operation of the cycling-posture analyzing system will be described hereinafter.

Specifically, the electronic device 11 comprises a transceiver 111 and a processor 113 which are electrically connected with each other. Four motion sensors 13a, 13b, 13c and 13d are disposed on a human body and are configured to detect respectively a plurality of pieces of motion information S1, S2, S3 and S4. One pressure sensor 15 is disposed at a plantar aspect of the human body and configured to detect pressure information Pl. The transceiver 111 is configured to receive the motion information S1-54 of all the motion sensors 13a to 13d and the pressure information P1 of the pressure sensor 15. Thereafter, the processor 113 determines cycling-posture type information N1 according to the motion information S1 to S4 and the pressure information P1 and on the basis of a cycling-posture identification model M1.

It shall be appreciated that, the electronic device 11 may be a mobile device, a smart device, a smart watch, a tablet computer, an earphone or an electronic product having the displaying function or the audio function. In an implementation, after determining the cycling-posture type information N1, the electronic device 11 may display the cycling-posture type information N1 on a screen, or provide the cycling-posture type information N1 to a user via an audio device. In some embodiments, the screen may be the screen of a mobile device or a smart watch, and in some other embodiments, the screen may also be the screen of another electronic device or an independent display, and it may be mounted on a bicycle.

The transceiver 111 is capable of wireless communication, and it may receive wireless signals of the motion sensors 13a to 13d and the pressure sensor 15, e.g., Wi-Fi signals, Bluetooth signals, device-to-device wireless signals or the like, and it may also transmit the cycling-posture type information N1 to any device requesting data via a wireless network. The processor 113 may be a combination of various processing units, central processing units (CPUs), microprocessors or various computing circuits, and it comprises a register or a memory, and the cycling-posture identification model M1 processes information in the register or the memory of the processor. In an implementation, the electronic device 11 may comprise a storage (not shown), which is configured to store an original cycling-posture identification model M1 and collect the cycling-posture type information N1. The storage may be a hard disk, a universal serial bus (USB) disk, a secure digital (SD) memory card, a mobile disk or other storage media or circuits.

It shall be appreciated that, the motion sensors 13a to 13d may be inertial measurement units (IMUs) which may be disposed, tied or worn at positions such as the waist, the thigh, the shank, or the foot of a human body. Additionally, the motion sensors 13a to 13d may be disposed, attached or bonded to cycling pants, cycling clothes or cycling shoes.

The pressure sensor 15 is disposed a plantar aspect of the human body, and it may be configured to sense plantar aspect pressure distribution information, e.g., pressure information at toes, the sole or the heel of the foot. The pressure sensor 15 may be combined with bicycle shoes. In an implementation, the cycling-posture analyzing system has two pressure sensors 15 disposed at the plantar aspect of two feet to measure a plurality of pieces of pressure information and obtain a more accurate analyzing result.

Moreover, the motion sensors 13a to 13d and the pressure sensor 15 of FIG. 1 are all capable of wireless signal transmission, and the numbers and disposing positions thereof are only used for illustration and are not limited thereto. Additionally, the motion sensors 13a to 13d and the pressure sensor 15 are only disposed at one of two lower limbs of the human body in the first embodiment, but they may also be disposed at both of the two limbs of the human body, and the design thereof shall be appreciated by those of ordinary skill in the art according to the above description.

It shall be appreciated that, the cycling-posture identification model M1 is a pre-established identification model which records a plurality of kinds of cycling-posture types and the corresponding limb movements and plantar aspect pressure distribution. The cycling-posture identification model M1 may be established by collecting a large amount of data accompanied with deep learning methods. Therefore, the cycling-posture identifying system may determine the current cycling-posture type of a rider according to the motion information S1 to S4, the pressure information P1 and the cycling-posture identification model M1.

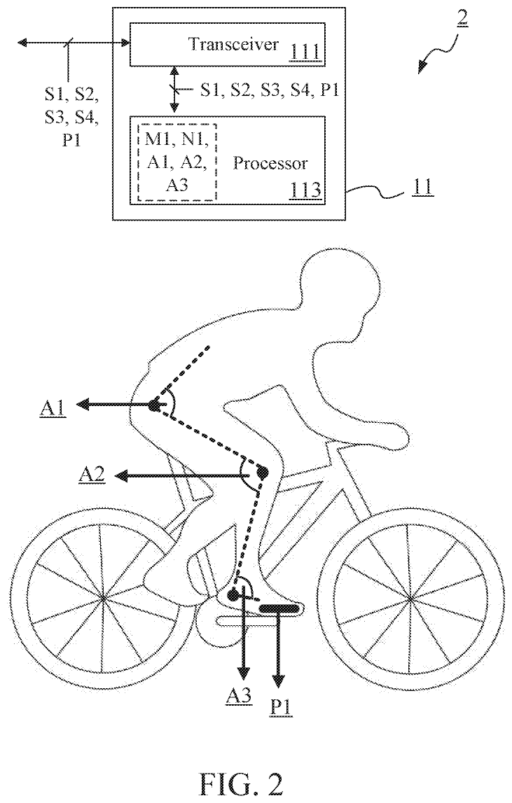

A second embodiment of the present invention is an extension of the first embodiment, and reference is made to FIG. 1 and FIG. 2 together for the second embodiment of the present invention. As shown in FIG. 2, elements and functions of the cycling-posture analyzing system 2 disclosed in the second embodiment are similar to these of the cycling-posture analyzing system 1 disclosed in the first embodiment, and thus will not be further described herein, and only differences therebetween will be detailed hereinafter. In the second embodiment, the processor 113 further calculates at least one piece of angular information according to the motion information S1 to S4. Thereafter, the processor 113 determines the cycling-posture type information N1 according to the at least one piece of angular information and the pressure information P1 and on the basis of the cycling-posture identification model M1.

In more detail, the processor 113 may calculate a piece of angular information A1 according to the motion information S1 and the motion information S2, may calculate a piece of angular information A2 according to the motion information S2 and the motion information S3, and may calculate a piece of angular information A3 according to the motion information S3 and the motion information S4. The cycling-posture identification model M1 further records relationships between cycling-posture types and the limb angles as well as plantar aspect pressures. Therefore, the processor 113 may determine the cycling-posture type information N1 according to at least one of the angular information A1 to A3 and the pressure information P1 and on the basis of the cycling-posture identification model M1.

A third embodiment of the present invention is an extension of the second embodiment, and reference is still made to FIG. 1 and FIG. 2 for the third embodiment of the present invention. Specifically, the third embodiment is a preferred implementation. The motion sensor 13a is disposed on a waist of the human body and is configured to detect the motion information S1 among the plurality of pieces of motion information. The motion sensor 13b is disposed on a thigh of the human body and is configured to detect the motion information S2 among the plurality of pieces of motion information. The motion sensor 13c is disposed on a shank of the human body and is configured to detect the motion information S3 among the plurality of pieces of motion information. The motion sensor 13d is disposed on an ankle of the human body and is configured to detect the motion information S4 among the plurality of pieces of motion information.

The processor 113 calculates the angular information A1 according to the motion information S1 and the motion information S2, calculates the angular information A2 according to the motion information S2 and the motion information S3, calculates the angular information A3 according to the motion information S3 and the motion information S4, and determines the cycling-posture type information N1 according to the angular information A1, the angular information A2, the angular information A3 and the pressure information P1 and on the basis of the cycling-posture identification model M1. In other words, a back-lowering angle, a knee joint angle and an ankle joint angle can be respectively obtained by calculating the angular information A1 to A3, and can be applied to subsequent analysis for analyzing the movement of lower limbs more accurately.

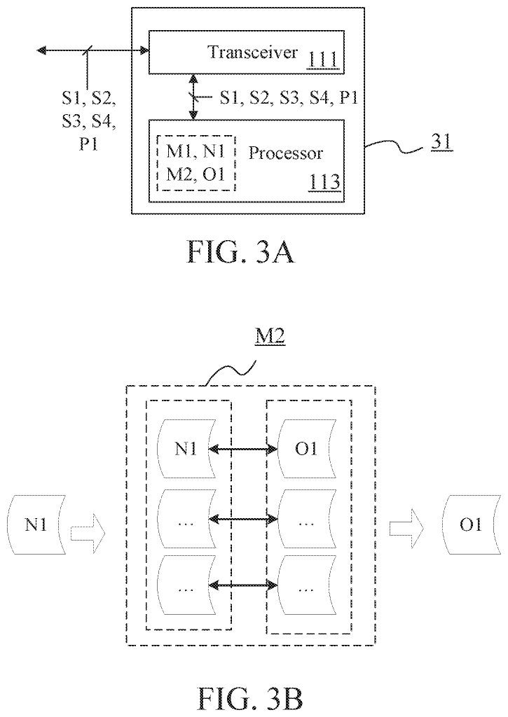

A fourth embodiment of the present invention is an extension of the first embodiment, and reference is made to FIG. 3A and FIG. 3B for the fourth embodiment of the present invention. FIG. 3A is an electronic device 31 of the fourth embodiment, and FIG. 3B is a schematic view depicting the operation of a muscle-group usage identification model M2 according to the fourth embodiment. Elements and functions of the cycling-posture analyzing system of the fourth embodiment are similar to these of the cycling-posture analyzing system of the first embodiment, and thus only differences therebetween will be detailed hereinafter.

Specifically, in the fourth embodiment, the processor 113 further determines muscle-group usage information O1 according to the cycling-posture type information N1 and on the basis of a muscle-group usage identification model M2, and wherein the muscle-group usage identification model M2 comprises pre-stored correspondence relationships between the cycling-posture type information and the muscle-group usage information.

In detail, the muscle-group usage identification model M2 may be established by electromyography experimental data according to laboratories to pre-establish the correspondence relationships between the cycling-posture types and the muscle-group usages. For different cycling-posture types, the hip positions and the cycling-posture types of the rider are all different, and different main muscle groups are applied. The motion information S1 to S4 and the pressure information P1 are first used to analyze the cycling-posture type information N1, i.e., the cycling-posture type of the bicycle rider. Thereafter, the current muscle-group usage information O1 of the rider is further determined according to the pre-defined muscle-group usage identification model M2.

For example, if the cycling-posture type is that the pelvic angle is neutral and natural, then the hip of the rider is located in the middle of the saddle, the cycling state feature is that the pedaling action only involves the stretching of the hip joint and a high-outputting strength can be maintained even after a long period of cycling, and the muscle group mainly used is the gluteus. If the cycling-posture type is that the pelvis tilts forward, then the hip of the rider is located in the front part of the saddle, the cycling state feature is that the angle of the knee joint becomes narrower, which tends to cause the front loading of the handlebars, and the muscle group mainly used is the quadriceps femoris. If the cycling-posture type is that the pelvis tilts backward, then the hip of the rider is located in the back part of the saddle, the cycling state feature is that the femoral joint is stretched using hamstring, one's own weight cannot be used during the pedaling, the pedaling frequency is hard to be increased, and muscle-group mainly used is the hamstring. Therefore, the muscle-group usage identification model M2 may be established by experimental data of actually measured EMG or sEMG.

In an implementation, the electronic device 31 may comprise a storage (not shown) that is configured to store the cycling-posture identification model M1, the muscle-group usage identification model M2, the cycling-posture type information N1 and the muscle-group usage information O1.

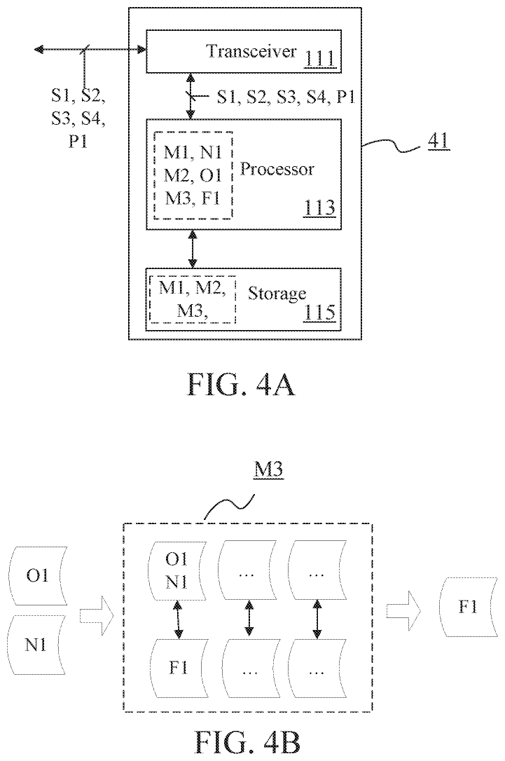

A fifth embodiment of the present invention is an extension of the fourth embodiment, and reference is made to FIG. 4A and FIG. 4B for the fifth embodiment of the present invention. FIG. 4A is an electronic device 41 of the fifth embodiment, and FIG. 4B is a schematic view depicting the usage of a feedback model M3 according to the fourth embodiment. Elements and functions of the cycling-posture analyzing system of the fifth embodiment are similar to these of the cycling-posture analyzing system of the fourth embodiment, and thus only differences therebetween will be detailed hereinafter.

In the fifth embodiment, the processor 113 is further configured to use the cycling-posture type information N1 and the muscle-group usage information O1 to decide feedback information F1 according to a feedback model M3, wherein the feedback information F1 is a piece of cycling-posture type suggestion information. In other words, the cycling-posture analyzing system may further provide a suitable cycling-posture to the rider according to the cycling-posture type information N1, the muscle-group usage information O1 and the feedback model M3, thereby prompting the rider to change the cycling-posture type. It shall be appreciated that, the feedback model M3 is pre-established in the cycling-posture analyzing system, and it comprises correspondence relationships between the cycling-posture type information and the muscle-group usage information and the feedback information.

In an implementation, the electronic device 41 further comprises a storage 115 which is electrically connected to the processor 113 and is configured to store one or a combination thereof. Moreover, the storage 115 may further comprise a database which is configured to store relevant data of the cycling-posture identification model M1, the muscle-group usage identification model M2 and the feedback model M3 so that the cycling-posture identifying system updates the cycling-posture identification model M1, the muscle-group usage identification model M2 and the feedback model M3, or updates the models by machine learning.

In an implementation, the cycling-posture type suggestion information is one of or a combination of cycling-posture type, cycling-posture type suggestion (i.e., suggestions for adjusting the cycling-posture), muscle-group usage suggestion (i.e., suggestions for focusing on the usage of specific muscle-group or suggestions for training another muscle-group) and hip to saddle position suggestion (i.e., suggestions for moving rider's hip from a position of the saddle to another position of the saddle). In other words, the cycling-posture type suggestion information may comprise suggestion data such as the suggested cycling-posture, the pedaling frequency, the pedaling force, the pedaling power, the time of duration, the center-of-gravity position of foot, the joint torque or the like.

In an implementation, the feedback model M3 has a plurality of kinds of cycling-posture type data and a suggestion rule. The feedback model M3 may decide that the cycling-posture type of the rider corresponds to one of the plurality kinds of the cycling-posture type data according to the cycling-posture type information N1 and the muscle-group usage information O1, the suggestion rule is configured to record at least one piece of cycling-posture suggestion information corresponding to each of the muscle-group usage information and a usage time of the muscle-group usage information, and the at least one piece of cycling-posture suggestion information is another kind one of the plurality kinds of the cycling-posture type data. In other words, after determining the cycling-posture type data corresponding to the current cycling-posture type, the feedback model M3 further decides another kind cycling-posture type data that is suitable for the subsequent cycling-posture type according to the muscle-group usage information O1 and the usage time of the muscle-group usage information O1, thereby providing a suggestion of changing the cycling-posture type to the user.

In an implementation, the establishment of the suggestion rule of the feedback model M3 is achieved by: (1) integrating multiple expert interviews and suggestions given on ordinary textbooks; (2) using expert assistance, analyzing a large amount of user data and machine learning; (3) calculating an ordinary optimal suggestion rule by the knowledge of anatomy and the principle of sport biomechanics, and establishing a suggestion rule customizing a feedback model based on the feedback after each cycling of the user via a method of machine learning; (4) making statistics on cycling strategies used by professional riders in long distance cycling by collecting a large amount of cycling data of multiple professional riders, and establishing a suggestion rule of a feedback model using machine learning; or (5) a combination of the aforesaid four parts.

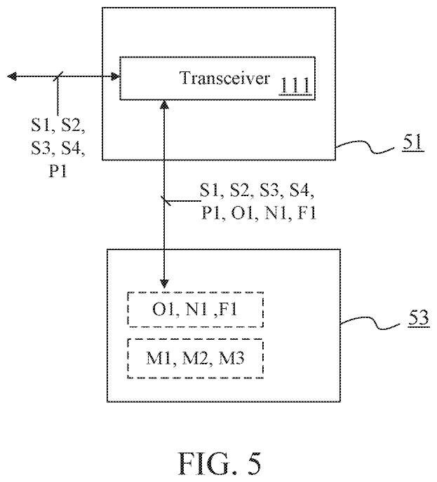

Please refer to FIG. 5 for a sixth embodiment of the present invention. FIG. 5 is a schematic view depicting an electronic device 51 of a cycling-posture analyzing system. Referring to FIG. 5, the sixth embodiment is an improvement of the fifth embodiment. Elements and functions of the cycling-posture analyzing system of the sixth embodiment are similar to these of the cycling-posture analyzing system of the fifth embodiment, and thus will not be further described herein, and only differences therebetween will be detailed hereinafter.

In the sixth embodiment, the electronic device 51 comprises a transceiver 111 which is connected with a cloud computing system 53 via a wireless network. The transceiver 111 is configured to receive the motion information S1 to S4 from the motion sensors 13a to 13d and the pressure information P1 from the pressure sensor 15. Thereafter, the transceiver 111 transmits the motion information S1 to S4 and the pressure information P1 to the cloud computing system 53 so that the cloud computing system 53 determines the cycling-posture type information N1, the muscle-group usage information O1 and the feedback information F1 according to the motion information S1 to S4 and the pressure information P1; and receive the cycling-posture type information N1, the muscle-group usage information O1 and the feedback information F1 from the cloud computing system 53.

More specifically, the electronic device 51 transmits the measured data to the cloud computing system 53 via the transceiver 111 without requiring the processor 113 for operation. The cloud computing system 53 stores the cycling-posture identification model M1, the muscle-group usage identification model M2 and the feedback model M3. The cloud computing system 53 may determine the cycling-posture type information N1 according to the motion information S1 to S4 and the pressure information P1 and on the basis of the cycling-posture identification model M1. The cloud computing system 53 may determine the muscle-group usage information O1 according to the cycling-posture type information N1 and on the basis of the muscle-group usage identification model M2. Thereafter, the cloud computing system 53 may decide the feedback information F1 according to the muscle-group usage information O1 and on the basis of the feedback model M3.

Moreover, in an implementation, the electronic device 51 may provide the cycling-posture type information N1, the muscle-group usage information O1 and/or the feedback information F1 to the user via a display device and/or an audio device. Additionally, in another implementation, the cloud computing system 53 may be a system having the operation function, such as a smart phone, a tablet computer or the like.

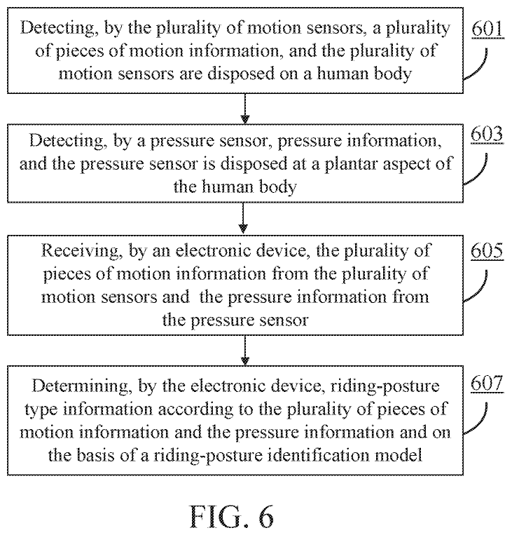

A seventh embodiment of the present invention is a cycling-posture analyzing method, and reference may be made to FIG. 6 for a flowchart diagram thereof. The method of the seventh embodiment is used for a cycling-posture analyzing system (e.g., the cycling-posture analyzing system of the aforesaid embodiments). The cycling-posture analyzing system comprises an electronic device, a plurality of motion sensors and a pressure sensor. The detailed steps of the seventh embodiment are as follows.

First, step 601 is executed to detect, by the plurality of motion sensors, a plurality of pieces of motion information, and the plurality of motion sensors are disposed on a human body. Step 603 is executed to detect, by the pressure sensor, pressure information, and the pressure sensor is disposed at a plantar aspect of the human body. Step 605 is executed to receive, by the electronic device, the plurality of pieces of motion information from the plurality of motion sensors and the pressure information from the pressure sensor. Step 607 is executed to determine, by the electronic device, cycling-posture type information according to the plurality of pieces of motion information and the pressure information and on the basis of a cycling-posture identification model.

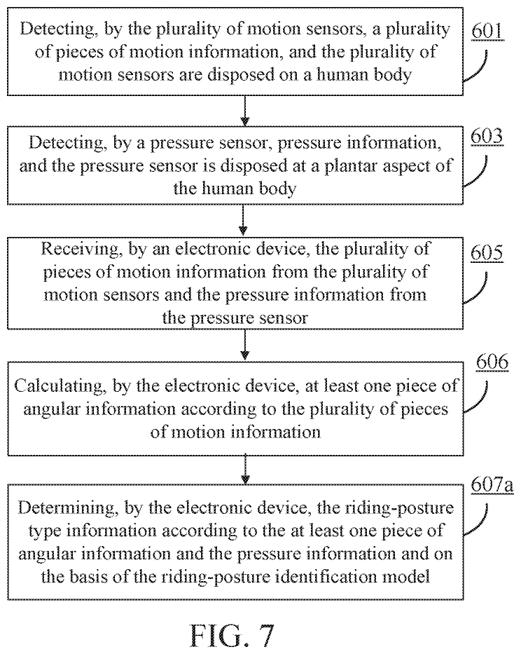

Please refer to FIG. 7 for an eighth embodiment of the present invention. The steps 601, 603 and 605 are similar to those of the seventh embodiment, and thus will not be further described herein.

After the step 605, the eighth embodiment further comprises step 606 to calculate, by the electronic device, at least one piece of angular information according to the plurality of pieces of motion information. Thereafter, step 607 further comprises step 607a to determine, by the electronic device, the cycling-posture type information according to the at least one piece of angular information and the pressure information and on the basis of the cycling-posture identification model.

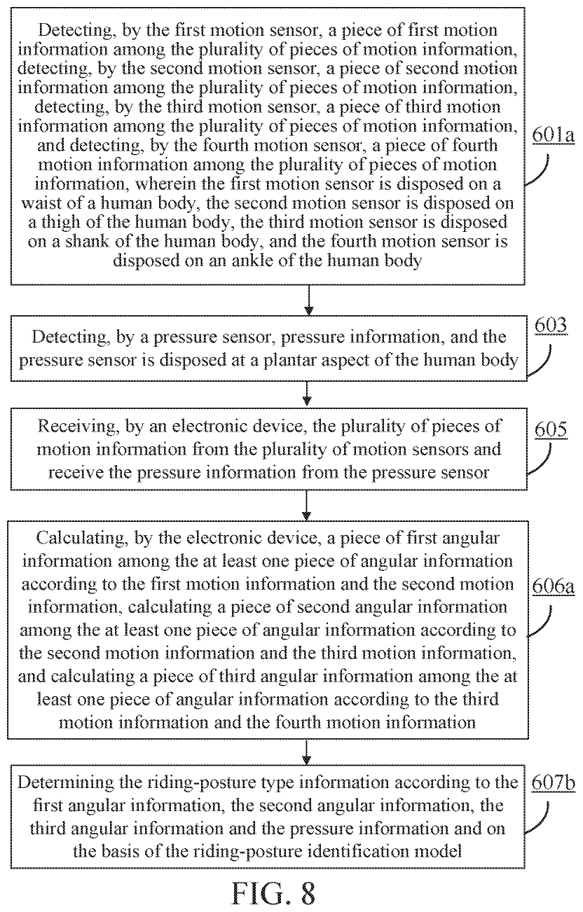

Please refer to FIG. 8 for a ninth embodiment of the present invention. In this implementation, the plurality of motion sensors further include a first motion sensor, a second motion sensor, a third motion sensor and a fourth motion sensor, wherein the first motion sensor is disposed on a waist of a human body, the second motion sensor is disposed on a thigh of the human body, the third motion sensor is disposed on a shank of the human body, and the fourth motion sensor is disposed on an ankle of the human body. Step 601a of detecting motion information detects, by the first motion sensor, a piece of first motion information among the plurality of pieces of motion information, detects, by the second motion sensor, a piece of second motion information among the plurality of pieces of motion information, detects, by the third motion sensor, a piece of third motion information among the plurality of pieces of motion information, and detects, by the fourth motion sensor, a piece of fourth motion information among the plurality of pieces of motion information. In some other embodiments, for example, the first motion sensor may be disposed on a waist of a human body, the second motion sensor may be disposed on a thigh of the human body, the third motion sensor may be disposed on a shank of the human body, and the fourth motion sensor may be disposed on an ankle of the human body. Step 603 is executed to detect, by the pressure sensor, pressure information, wherein the pressure sensor is disposed at a plantar aspect of the human body. Step 605 is executed to receive, by the electronic device, the plurality of pieces of motion information from the plurality of motion sensors and the pressure information from the pressure sensor.

Thereafter, the step of calculating at least one piece of angular information further comprises step 606a of: calculating, by the electronic device, a piece of first angular information among the at least one piece of angular information according to the first motion information and the second motion information, calculating, by the electronic device, a piece of second angular information among the at least one piece of angular information according to the second motion information and the third motion information, and calculating, by the electronic device, a piece of third angular information among the at least one piece of angular information according to the third motion information and the fourth motion information.

The step 607a further comprises step 607b of determining the cycling-posture type information according to the first angular information, the second angular information, the third angular information and the pressure information and on the basis of the cycling-posture identification model.

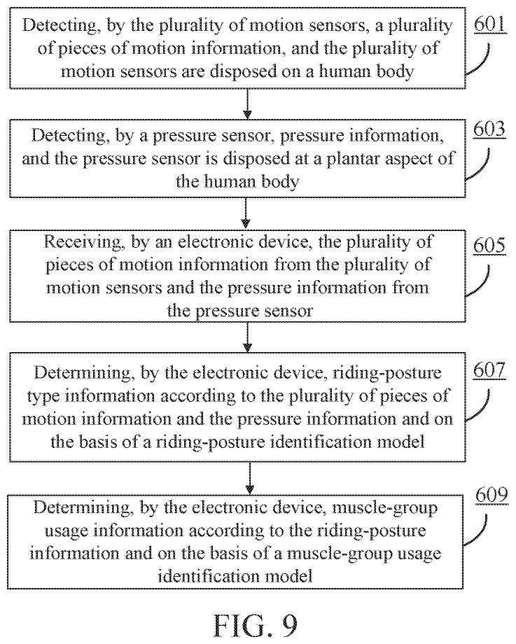

Please refer to FIG. 9 for a tenth embodiment of the present invention, which is an extension of the seventh embodiment. The flow process of the tenth embodiment is similar to that of the seventh embodiment, and thus the same contents will not be repeated herein and the following description will focus on the differences therebetween.

Specifically, after the step 607, the cycling-posture analyzing method further comprises step 609 of determining, by the electronic device, muscle-group usage information according to the cycling-posture type information and on the basis of a muscle-group usage identification model.

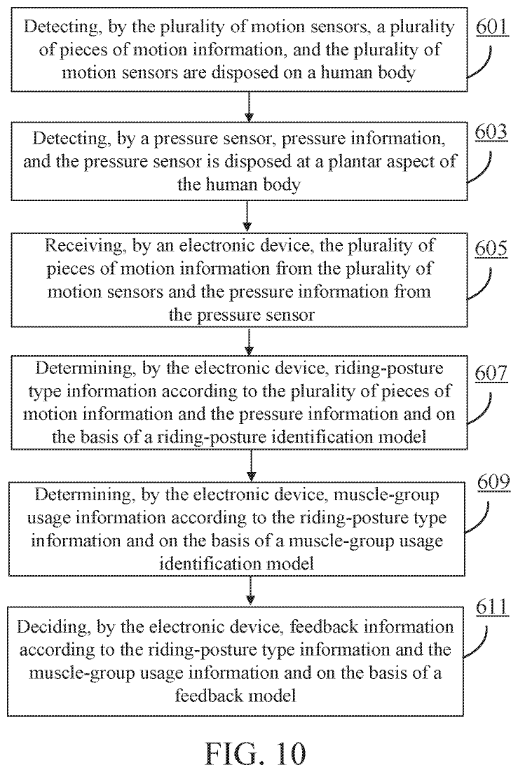

Please refer to FIG. 10 for an eleventh embodiment of the present invention, which is an extension of the tenth embodiment. The flow process of the eleventh embodiment is similar to that of the tenth embodiment, and thus the same contents will not be repeated herein and the following description will focus on the differences therebetween.

Specifically, after the step 609, the cycling-posture analyzing method further comprises step 611 of deciding, by the electronic device, feedback information according to the cycling-posture type information and the muscle-group usage information and on the basis of a feedback model.

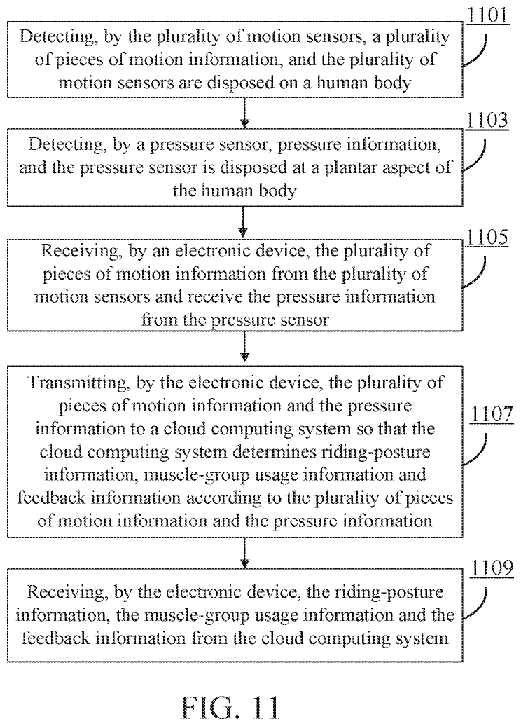

A twelfth embodiment of the present invention is a cycling-posture analyzing method, and reference may be made to FIG. 11 for a flowchart diagram thereof. The method of the twelfth embodiment is used for a cycling-posture analyzing system (e.g., the cycling-posture analyzing system of the aforesaid embodiments). The cycling-posture analyzing system comprises an electronic device, a plurality of motion sensors and a pressure sensor. The detailed steps of the twelfth embodiment are as follows.

First, step 1101 is executed to detect, by the plurality of motion sensors, a plurality of pieces of motion information, and the plurality of motion sensors are disposed on a human body. Step 1103 is executed to detect, by the pressure sensor, pressure information, and the pressure sensor is disposed at a plantar aspect of the human body. Step 1105 is executed to receive, by the electronic device, the plurality of pieces of motion information from the plurality of motion sensors and the pressure information from the pressure sensor.

Step 1107 is executed to transmit, by the electronic device, the plurality of pieces of motion information and the pressure information to a cloud computing system so that the cloud computing system determines cycling-posture type information, muscle-group usage information and feedback information according to the plurality of pieces of motion information and the pressure information. Step 1107 is executed to receive, by the electronic device, to receive the cycling-posture type information, the muscle-group usage information and the feedback information from the cloud computing system.

In addition to the aforesaid seventh embodiment to twelfth embodiment, the cycling-posture analyzing method of the present invention can also execute all the functions of the cycling-posture analyzing system of the first embodiment to the sixth embodiment of the present invention and achieve the same technical effects, and this will not be further described herein. Moreover, the aforesaid embodiments and implementations can be combined into one embodiment if the technical contents thereof are not conflicting with each other.

As can be known from the above descriptions, the cycling-posture analyzing system and the cycling-posture analyzing method provided according to the present invention perform detection and then determine the cycling-posture type information of the rider according to the motion information and the pressure information and on the basis of the cycling-posture identification model. Moreover, the system and the method can further provide the user with the muscle-group usage information and the feedback information based on the muscle-group usage identification model and the feedback model. The rider can receive the cycling-posture suggestion and accordingly adjust the current cycling-posture type during the cycling. Therefore, as compared to the prior art, the present invention can more effectively provide the rider with relevant cycling information during the outdoor cycling.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A cycling-posture analyzing system, comprising:
a plurality of motion sensors disposed on a human body, being configured to detect a plurality of pieces of motion information;
a pressure sensor disposed at a plantar aspect of the human body, being configured to detect pressure information; and
an electronic device, comprising:
a transceiver configured to receive the plurality of pieces of motion information from the plurality of motion sensors and the pressure information from the pressure sensor; and
a processor electrically connected to the transceiver, being configured to determine cycling-posture type information according to the plurality of pieces of motion information and the pressure information and on the basis of a cycling-posture identification model.

2. The cycling-posture analyzing system of claim 1, wherein the processor is further configured to:
calculate at least one piece of angular information according to the plurality of pieces of motion information;
determine the cycling-posture type information according to the at least one piece of angular information and the pressure information and on the basis of the cycling-posture identification model.

3. The cycling-posture analyzing system of claim 2, wherein the plurality of motion sensors include:
a first motion sensor disposed on a waist of the human body, configured to detect a piece of first motion information among the plurality of pieces of motion information;
a second motion sensor disposed on a thigh of the human body, configured to detect a piece of second motion information among the plurality of pieces of motion information;
a third motion sensor disposed on a shank of the human body, configured to detect a piece of third motion information among the plurality of pieces of motion information; and
a fourth motion sensor disposed on an ankle of the human body, configured to detect a piece of fourth motion information among the plurality of pieces of motion information;
wherein the processor is further configured to:
calculate a piece of first angular information among the at least one piece of angular information according to the first motion information and the second motion information;
calculate a piece of second angular information among the at least one piece of angular information according to the second motion information and the third motion information;
calculate a piece of third angular information among the at least one piece of angular information according to the third motion information and the fourth motion information; and
determine the cycling-posture type information according to the first angular information, the second angular information, the third angular information and the pressure information and on the basis of the cycling-posture identification model.

4. The cycling-posture analyzing system of claim 1, wherein the processor is further configured to:
determine muscle-group usage information according to the cycling-posture type information and on the basis of a muscle-group usage identification model.

5. The cycling-posture analyzing system of claim 4, wherein the processor is further configured to:
decide feedback information according to the cycling-posture type information and the muscle-group usage information and on the basis of a feedback model, wherein the feedback information shows a piece of cycling-posture type suggestion information.

6. The cycling-posture analyzing system of claim 1, wherein the plurality of motion sensors are inertial measurement units (IMUs).

7. The cycling-posture analyzing system of claim 5, wherein the electronic device further comprises:
a storage configured to store the cycling-posture identification model, the muscle-group usage identification model and the feedback model.

8. The cycling-posture analyzing system of claim 5, wherein the feedback model has a plurality kinds of cycling-posture type data and a suggestion rule, the cycling-posture type information and the muscle-group usage information correspond to one of the plurality kinds of cycling-posture type data, the suggestion rule records at least one piece of cycling-posture type suggestion information corresponding to each of the pieces of muscle-group usage information and a usage time of the piece of muscle-group usage information, and the at least one piece of cycling-posture type suggestion information is another kind of the plurality of cycling-posture type data.

9. The cycling-posture analyzing system of claim 5, wherein the cycling-posture suggestion information is one of cycling-posture type, cycling-posture type suggestion, muscle-group usage suggestion and saddle-cushion position suggestion.

10. A cycling-posture analyzing system, comprising:
a plurality of motion sensors disposed on a human body, being configured to detect a plurality of pieces of motion information;
a pressure sensor disposed at a plantar aspect of the human body, being configured to detect pressure information; and
an electronic device, comprising:
a transceiver, being configured to:
receive the plurality of pieces of motion information from the plurality of motion sensors and the pressure information from the pressure sensor;
transmit the plurality of pieces of motion information and the pressure information to a cloud computing system so that the cloud computing system determines cycling-posture type information, muscle-group usage information and feedback information according to the plurality of pieces of motion information and the pressure information; and
receive the cycling-posture type information, the muscle-group usage information and the feedback information from the cloud computing system.

11. A cycling-posture analyzing method for a cycling-posture analyzing system, the cycling-posture analyzing system comprising a plurality of motion sensors, a pressure sensor and an electronic device, and the cycling-posture analyzing method comprising:
detecting, by the plurality of motion sensors, a plurality of pieces of motion information, the plurality of motion sensors being disposed on a human body;
detecting, by the pressure sensor, pressure information, the pressure sensor being disposed at a plantar aspect of the human body;
receiving, by the electronic device, the plurality of pieces of motion information from the plurality of motion sensors and receive the pressure information from the pressure sensor; and
determining, by the electronic device, cycling-posture type information and on the basis of the plurality of pieces of motion information and the pressure information and on the basis of a cycling-posture identification model.

12. The cycling-posture analyzing method of claim 11, further comprising:
calculating, by the electronic device, at least one piece of angular information according to the plurality of pieces of motion information;
determining, by the electronic device, the cycling-posture type information and on the basis of the at least one piece of angular information and the pressure information and on the basis of the cycling-posture identification model.

13. The cycling-posture analyzing method of claim 12, wherein the plurality of motion sensors further include a first motion sensor, a second motion sensor, a third motion sensor and a fourth motion sensor, and the step of detecting the plurality of pieces of motion information further comprises:
detecting, by the first motion sensor, a piece of first motion information among the plurality of pieces of motion information, the first motion sensor being disposed on a waist of the human body;
detecting, by the second motion sensor, a piece of second motion information among the plurality of pieces of motion information, the second motion sensor being disposed on a thigh of the human body;
detecting, by the third motion sensor, a piece of third motion information among the plurality of pieces of motion information, the third motion sensor being disposed on a shank of the human body; and
detecting, by the fourth motion sensor, a piece of fourth motion information among the plurality of pieces of motion information, the fourth motion sensor being disposed on an ankle of the human body;
wherein the step of calculating the at least one piece of angular information further comprises:
calculating, by the electronic device, a piece of first angular information among the at least one piece of angular information according to the first motion information and the second motion information;
calculating, by the electronic device, a piece of second angular information among the at least one piece of angular information according to the second motion information and the third motion information;
calculating, by the electronic device, a piece of third angular information among the at least one piece of angular information according to the third motion information and the fourth motion information; and
determining, by the electronic device, the cycling-posture type information according to the first angular information, the second angular information, the third angular information and the pressure information and on the basis of the cycling-posture identification model.

14. The cycling-posture analyzing method of claim 11, further comprising:
determining, by the electronic device, muscle-group usage information according to the cycling-posture type information and on the basis of a muscle-group usage identification model.

15. The cycling-posture analyzing method of claim 14, further comprising:
deciding, by the electronic device, feedback information according to the cycling-posture type information and the muscle-group usage information and on the basis of a feedback model, wherein the feedback information shows a suggestion for cycling-posture.

16. The cycling-posture analyzing method of claim 15, wherein the feedback model has a plurality kinds of cycling-posture type data and a suggestion rule, the cycling-posture type information and the muscle-group usage information correspond to one of the plurality kinds of cycling-posture type data, the suggestion rule records at least one piece of cycling-posture suggestion information corresponding to each of the pieces of muscle-group usage information and a usage time of the piece of muscle-group usage information, and the at least one piece of cycling-posture suggestion information is another kind of the plurality of cycling-posture type data.

17. The cycling-posture analyzing method of claim 15, wherein the cycling-posture suggestion information is one of cycling-posture type, cycling-posture type suggestion, muscle-group usage suggestion and saddle-cushion position suggestion.
